(19) United States
(12) Patent Application Publication
(10) Pub. No.: US 2025/0198139 A1
(43) Pub. Date: Jun. 19, 2025

(54) FRAGRANCE BATH AND SHOWER FAUCET STRUCTURE

(71) Applicant: CROWN PRINCE SANITARY ENTERPRISE CO., LTD., Changhua County (TW)

(72) Inventor: Zong-Xian HE, Changhua County (TW)

(21) Appl. No.: 18/539,756

(22) Filed: Dec. 14, 2023

(51) Int. Cl. E03C 1/04; A61L 9/14; B05B 1/04; B05B 17/06

(52) U.S. Cl. CPC E03C 1/0408; A61L 9/14; B05B 1/044; B05B 17/0607; E03C 1/0403; A61L 2209/11; A61L 2209/134

(57) ABSTRACT

A fragrance bath and shower faucet structure with fragrance spray and water supply functions includes cold and hot water pipes, a shower head, a waterfall pipe, and a micro generator. Cold and hot water are passed through the micro generator to generate and store electric power in a battery to be supplied to a spray vibration motor on the base. A temperature adjustment knob is provided for adjusting the cold and hot water mixing ratio, and water temperature is indicated by lighting. The base is provided with three press buttons for controlling the water discharged from the faucet, a shower head water output mode and a fragrance spray function, and the base is covered with a lining board which is covered by a top cover. The lining board has a container for containing fragrance oil, and the top cover has a slot for setting up a decorative object.

### BACKGROUND OF THE DISCLOSURE

### Technical Field

The present disclosure relates to a fragrance bath and shower faucet structure, more particularly relates to a bath and shower faucet based on the functional structure of existing bath and shower faucets, integrated with a fragrance spray function by structural planning and configuration, and using the water from a faucet to generate and store electric power and supply the electric power required by the operation of a spray vibration motor for the fragrance spray, which in turn makes the faucet to have an independent fragrance spray function and overcomes the problem and inconvenience caused by the replacement of battery.

### Description of the Related Art

Traditional bath faucets are usually designed with two different water output modes including a faucet water output mode and a shower head water output mode, and a lever or toggle switch is provided to switch the water output mode to meet different water needs of the users.

With the improvement of the quality of life, the existing shower faucets are no longer designed with an external appearance purely for their function only, but shower faucets, bathtubs and other sanitary ware are designed for their collocation instead, so that the overall stylish and visual appearance has better harmony. However, no matter how the appearance of the faucet is changed, the function of the faucet is still limited to two different water output modes: the faucet water output mode and shower head water output mode.

As to this type of bath and shower faucets, the faucet is usually installed at a predetermined position of a bathtub or around the bathtub, so water can be injected into the bathtub to meet the bathing requirement. By the shower head water output mode, the need of outputting water for shower or rinsing the bathroom can be met. Taking a bubble bath for example, we usually immerse our body in water at an appropriate temperature to achieve the effects of relaxation and blood circulation, so such bath will usually take a longer time. In order to create a comfortable environment for the bubble bath, floral fragrance such as lavender or other fragrance is usually used, and the essence oil released from the fragrance oil gives a calming effect.

At present, the effect of providing the fragrance is usually achieved by installing another fragrance spray machine, and the fragrance spray machine includes a built-in spray vibration motor that generate a vibration frequency to let the essential oil be atomized, so as to achieve the effect of the fragrance. This type of fragrance spray machine usually adopts the mains power or dry battery to supply power, but the mains power used in the humid bathroom may easily cause short-circuits or give rise to safety concerns. The use of dry cell batteries has the trouble and inconvenience of replacing the battery when it is exhausted. In addition, the dry cell batteries need independent set-up and operation, not only wasting space, but also causing inconvenience to operate. Obviously, the related art requires improvements.

### SUMMARY OF THE DISCLOSURE

In view of the drawbacks and inconvenience of the conventional bath and shower faucet and fragrance spray machine that the separate stand-alone configuration not only has tremendous trouble and inconvenience in configuration and use, but also has difficulties to achieve harmony. Especially, there are issues of safety of use and replacement of battery for the additional power supply required by the fragrance spray machine. Based on these drawbacks of the prior art, the present discloser based on years of experience to conduct research and experiment, and finally developed a fragrance bath and shower faucet structure in accordance with the present disclosure.

The present disclosure discloses a structural design of a fragrance bath and shower faucet, which mainly guides the cold and hot water pipes of the faucet through a micro generator, and then mixes the cold water and the hot water and adjusts the water temperature before selectively supplying water in a faucet water output mode or a shower head water output mode, and uses the micro generator to generate and store electric power in a battery when water flows through the micro generator, and finally supplies the electric power of the battery through an integrated circuit on the side to a spray vibration motor to produce high-frequency vibrations for the essential oil contained in the corresponding part of the motor when the spray vibration motor is activated, so as to achieve the fragrance spray effect and integrate the fragrance spray function with the faucet water supply function.

In the structural design of the fragrance bath faucet of the present disclosure, the fragrance spray function is integrated into the faucet, and the use of the water outputted from the faucet is provided to drive the micro-generator to generate electric power and store the electric power in the battery, which is supplied for the operation of the spray vibration motor, so that in addition to the function of the fragrance spray, the replacement of dry batteries or the configuration of additional power line can be exempted to provide a more practical and convenient application.

### BRIEF DESCRIPTION OF THE DRAWINGS

FIG. 1 is a perspective view of a preferred embodiment of the present disclosure;

FIG. 2 is a perspective view of a preferred embodiment of the present disclosure, viewing from another direction;

FIG. 3 is a schematic view showing the structural disassembly of a preferred embodiment of the present disclosure;

FIG. 4 is a schematic view showing the internal structure configuration of a preferred embodiment of the present disclosure; and

FIG. 5 is a schematic view showing an implementation mode of a preferred embodiment of the present disclosure.

### DESCRIPTION OF THE PREFERRED EMBODIMENTS

The structural assembly, technical measures and effects of the present disclosure will become apparent in the following detailed description of the preferred embodiments with reference to the accompanying drawings.

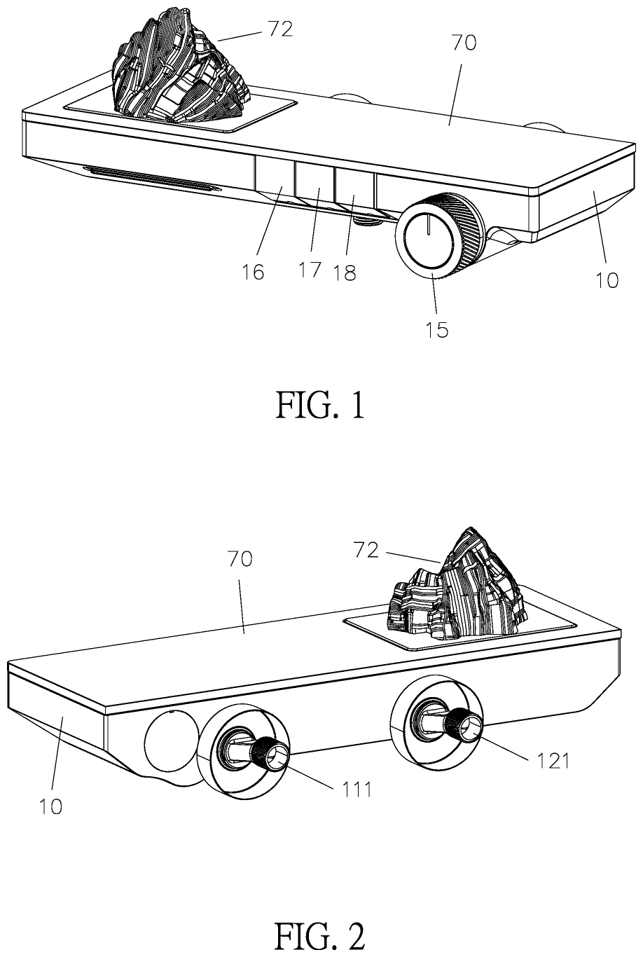

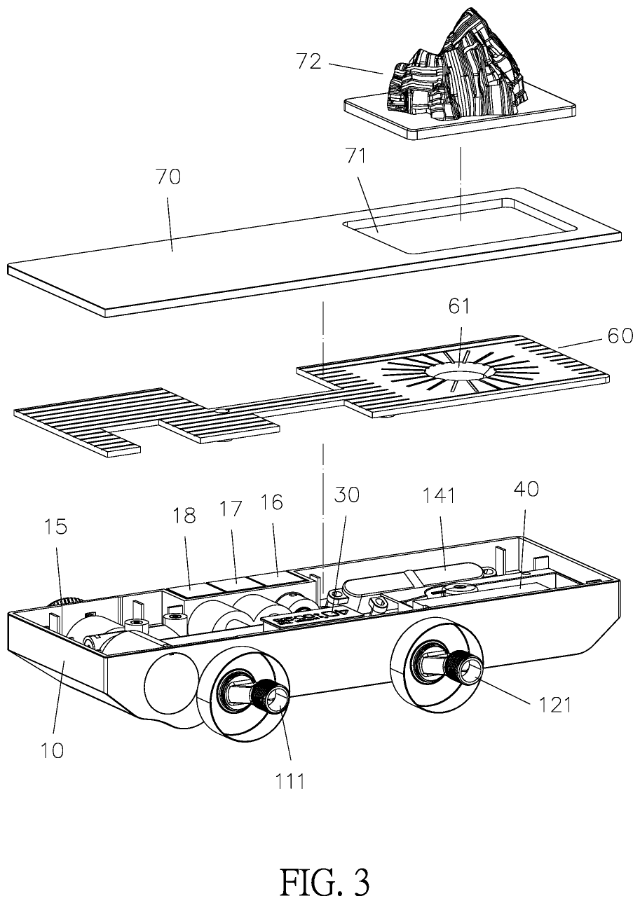

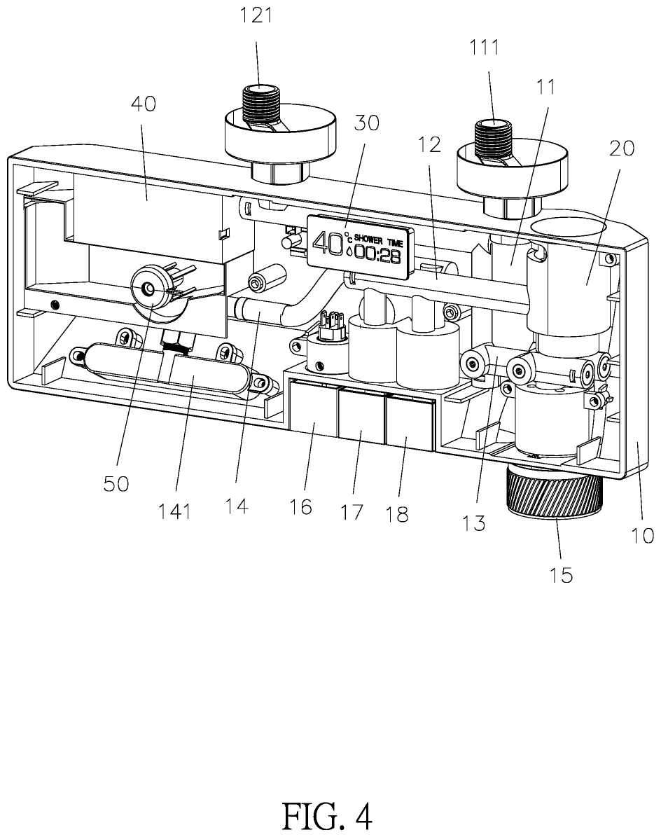

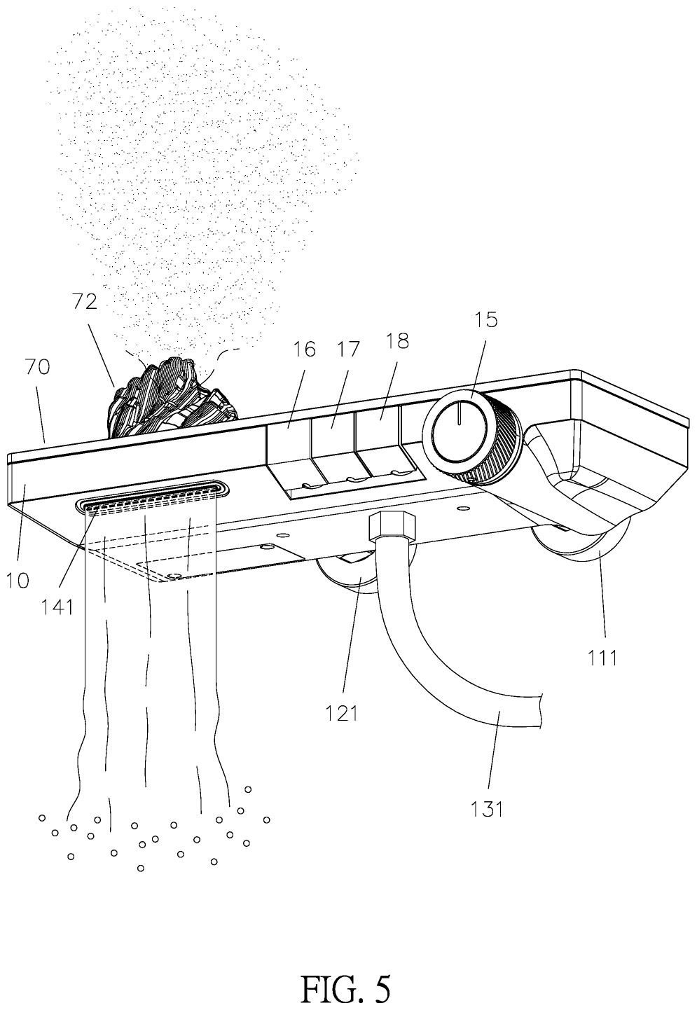

With reference to FIGS. 1 to 5 for the implementation mode in accordance with a preferred embodiment of the present disclosure, the present disclosure provides a structural design of a fragrance bath and shower faucet which includes a stylish base (10), cold and hot water pipes (11) (12), a shower head water outlet pipe (13), and a faucet water outlet pipe (14) which are arranged inside the base (10). The shower head water outlet pipe (13) is externally connected to a shower head hose (131), the faucet water outlet pipe (14) is connected to a horizontal outlet head (141) and installed at the front end of a side of the base (10) to provide a waterfall-like water output mode. In addition, the cold and hot water pipes (11) (12) are provided with a pipe connector (111) (121) extended towards the backside of the base (10) and exposed to the outside, and connected to the cold and hot water pipes on a wall surface, and predetermined positions of a side of the base (10) is provided with a plurality of temperature adjustment knobs (15) for adjusting the cold and hot water mixing ratio, and the status of water temperature is indicated by a lighting effect. In order to integrate the fragrance spray function into the bath and shower faucet, the water inlet path of the cold and hot water pipes (11) (12) of the present disclosure is equipped with a micro generator (20) for guiding the cold and hot water into the faucet to generate electric power by the micro generator (20), and the electric power is stored in a battery (30) installed at a predetermined position adjacent to the base (10), and the battery (30) in conjunction with an integrated circuit (40) supply the electric power to a spray vibration motor (50) installed at a predetermined position of the base (10). In addition, the front side of the base (10) is provided with three press buttons (16) (17) (18) for controlling the water discharged from the faucet, a shower head water output mode and a fragrance spray function respectively, and the top of the base (10) is covered with a lining board (60) for hiding the aforementioned structure, and the top of the lining board (60) is covered with a top cover (70) to seal the base (10) and complete the overall assembly. In addition, the lining board (60) has a container (61) configured to be corresponsive to the spray vibration motor (50) and provided for pouring and containing fragrance oils. During the operation of the spray vibration motor (50), its high-frequency vibration will atomize and disperse the essential oil in the container (61), and the top cover (70) has a slot (71) formed at a position corresponding to the container (61) on the lining board (60) and provided for setting a decorative object (72) such as rockery landscaping, etc. to create an overall visual quality, and these components constitute the assembly of the fragrance bath and shower faucet structure.

With the above structural design, the three press buttons (16) (17) (18) disposed on a side of the base (10) are provided for controlling and selecting two different water output modes including a faucet water output mode and a shower head water output mode, and determining whether or not to enable the fragrance spray function. Regardless of the selection of the water output mode, when the cold and hot water is inputted and passed through the micro generator (20), the micro generator (20) will generate electric power on its own and store the electric power in the battery (30) on a side as the power required for the operation of the spray vibration motor (50). After the fragrance spray function is enabled, the electric power stored in the battery (30) will be supplied to the spray vibration motor (50) for its operation, so as to achieve the expected fragrance spray function. To achieve this function, there will be no external mains wiring required, or replacement of dry cell battery. Obviously, the present disclosure provides a very practical and convenient application and integrates different functions into the existing bath and shower faucet structure. It surely enhances the overall visual quality and convenience of assembly, and effectively overcome the problems and drawbacks arising from the conventional separate configuration, which can be regarded as an outstanding innovative design.

In summation of the description above, the design of the fragrance bath and shower faucet of the present disclosure integrates the fragrance spray function into the existing faucet structure and uses the water inputted into the faucet to trigger the micro generator to generate electric power and store the electric power in a battery to be supplied to the spray vibration motor, so as to exempt the mains wiring, and trouble and inconvenience of the replacement of the dry cell battery. Obviously, the present disclosure is an innovative design that complies with patent application requirements, and thus is duly filed for patent application.

What is claimed is:

1. A fragrance bath and shower faucet structure, comprising a stylish base, a cold water pipe and a hot water pipes designed in the base, a shower head water outlet pipe, and a faucet water outlet pipe, and the cold and hot water pipes comprising a pipe connector extended towards and exposed from the backside of the base, for coupling the cold and hot water pipes on a wall surface, a plurality of temperature adjustment knobs disposed at predetermined positions on a side of the base respectively for adjusting a cold and hot water mixing ratio and displaying a water temperature status by a lighting effect, and a press button for selectively controlling a shower head water output mode or a faucet water output mode; characterized in that:
the cold and hot water pipes have a water inlet path equipped with a micro generator and drives the micro generator to generate and store electric power in a battery installed at a predetermined position adjacent to the base, the battery is paired with an integrated circuit and provided for supplying electric power to a spray vibration motor installed at a predetermined position of the base, the top of the base is covered with a lining board, and a top cover is covered onto the lining board to seal the base, the lining board is provided with a container configured to be corresponsive to the spray vibration motor and provided for pouring and containing a fragrance oil, and the spray vibration motor is operated to atomize and disperse the essential oil in the container, and a stylish decorative object is disposed at a position on the top cover and configured to be corresponsive to the container of the lining board, so as to integrate the fragrance spray function into the bath and shower faucet.

2. The fragrance bath and shower faucet structure according to claim 1, wherein the top cover comprises a slot configured to be corresponsive to the container of the lining board and provided for installing a rockery landscaping decorative object.

3. The fragrance bath and shower faucet structure according to claim 1, wherein the faucet water outlet pipe is coupled to a horizontal outlet head and installed to the front edge of a side of the base to give the waterfall-like water output mode.

4. The fragrance bath and shower faucet structure according to claim 1, wherein the base is equipped with three press buttons at the front end thereof, and the three press buttons are provided for controlling the faucet water output, the shower head water output, and the fragrance spray function respectively.
